(19) United States
(12) Patent Application Publication
(10) Pub. No.: US 2010/0318170 A1
(43) Pub. Date: Dec. 16, 2010

(54) PROXIMAL CATHETER FLAP FOR MANAGING WIRE TWIST

(76) Inventor: Richard Newhauser, Redwood City, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 12/484,869

(22) Filed: Jun. 15, 2009

(51) Int. Cl. A61F 2/06

(52) U.S. Cl. 623/1.11; 623/1.35; 623/1.46

(57) ABSTRACT

A stent delivery system for treating a diseased bifurcation vessel having a catheter with a securement feature. The delivery system includes a dual lumen catheter with one or more balloons and a specialized stent having a side port mounted on the balloon. The securement feature is a flap that retains a side branch guide wire near the catheter body and wherein the distal tip of the guide wire is positioned near the side port of the stent. At the bifurcation, the side branch guide wire is freed from the flap to advance into the side branch vessel through the stent side port.

### FIELD OF THE INVENTION

The invention relates to a method and apparatus for delivery and implantation of stents. More particularly, the invention relates to a catheter and stent delivery and deployment assemblies for use in repairing bifurcations and blood vessels that are diseased.

### BACKGROUND OF THE INVENTION

Stents conventionally repair blood vessels that are diseased. Stents are generally hollow and cylindrical in shape and have terminal ends that are generally perpendicular to their longitudinal axis. In use, the conventional stent is positioned at the diseased area of a vessel and, after deployment, the stent provides an unobstructed pathway for blood flow.

Repair of vessels that are diseased at a bifurcation is particularly challenging since the stent must be precisely positioned, provide adequate coverage of the lesion, provide access to any diseased area located distal to the bifurcation, and maintain vessel patency in order to allow adequate blood flow to reach the myocardium. Therefore, the stent must provide adequate coverage to the diseased portion of the bifurcated vessel, without compromising blood flow, and extend to a point within and beyond the diseased portion. Where the stent provides coverage to the vessel at the diseased portion, yet extends into the vessel lumen at the bifurcation, the diseased area is repaired, but blood flow may be compromised in other portions of the bifurcation. Unapposed stent elements may promote lumen compromise during neointimal formation and healing, producing restenosis and requiring further procedures. Moreover, by extending into the vessel lumen at the bifurcation, the stent may block access to further interventional procedures.

Conventional stents are designed to repair areas of blood vessels that are removed from bifurcations, and, therefore are associated with a variety of problems when attempting to use them to treat lesions at a bifurcation. Conventional stents are normally deployed so that the entire stent is either in the parent vessel or the proximal portion of the stent is in the parent vessel and the distal portion is located in the side branch vessel. In both cases, either the side branch vessel (former case) or the parent vessel (latter case), would become “jailed” by the stent struts. This technique repairs one vessel at the bifurcation at the expense of jailing or obstructing the alternate vessel. Blood flow into the jailed vessel would be diminished as well as future access and treatment into the distal portion of the jailed vessel.

Alternatively, access into a jailed vessel can be attained by carefully placing a guide wire through the stent, and subsequently tracking a balloon catheter through the stent struts. The balloon could then be expanded, thereby deforming the stent struts and forming an opening into the previously jailed vessel. The cell to be spread apart must be randomly and blindly selected by re-crossing the deployed stent with a guide wire. The drawback with this approach is that there is no way to determine or guarantee that the main vessel stent struts are properly oriented with respect to the side branch or that an appropriate stent cell has been selected by the wire for dilatation. The aperture created often does not provide a clear opening and creates a major distortion in the surrounding stent struts. A further drawback with this approach is that there is no way to tell if the main-vessel stent struts have been properly oriented and spread apart to provide a clear opening for stenting the side branch vessel. This technique also causes stent deformation to occur in the area adjacent to the carina, pulling the stent away from the vessel wall and partially obstructing flow in the originally non-jailed vessel. Deforming the stent struts to regain access into the previously jailed strut is also a complicated and time consuming procedure associated with attendant risks to the patient and is typically performed only if considered an absolute necessity. Vessels which supply a considerable amount of blood supply to the myocardium and may be responsible for the onset of angina or a myocardial infarction would necessitate subsequent strut deformation to reestablish blood flow into the vessel. The risks of procedural complications during this subsequent deformation are considerably higher than stenting in normal vessels. The inability to place a guide wire through the jailed lumen in a timely fashion could restrict blood supply and begin to precipitate symptoms of angina or even cardiac arrest. In addition, platelet agitation and subsequent thrombus formation at the jailed site could further compromise blood flow into the side branch.

Plaque shift is a phenomena which is of concern when deploying a stent across a bifurcation. Plaque shift occurs when treatment of disease or plaque in one vessel causes the plaque to shift into another location. This is of greatest concern when the plaque is located on the carina or the apex of the bifurcation. During treatment of the disease the plaque may shift from one side of the carina to the other thereby shifting the obstruction from one vessel to the alternate vessel.

Another method of implanting stents is a “T” stent procedure, which includes implanting a stent in the side branch ostium of the bifurcation followed by stenting the main vessel across the side branch and subsequently deforming the struts as previously described, to allow blood flow and access into the side branch vessel. Alternatively, a stent is deployed in the parent vessel and across the side branch origin followed by subsequent strut deformation as previously described, and finally a stent is placed into the side branch vessel. T stenting may be necessary in some situations in order to provide further treatment and additional stenting in the side branch vessel. This is typically necessitated when the disease is concentrated at the origin of the jailed vessel. This procedure is also associated with the same issues and risks previously described when stenting only one vessel and deforming the struts through the jailed vessel. In addition, since a conventional stent generally terminates at right angles to its longitudinal axis, the use of conventional stents to treat the origin of the previously jailed vessel (typically the side branch vessel) may result in blocking blood flow of the originally non-jailed vessel (typically the parent vessel) or failure to provide adequate coverage of the diseased area in the previously jailed vessel (typically a side branch vessel). The conventional stent might be placed proximally in order to provide full coverage around the entire circumference of the side branch. However, this leads to a portion of the stent extending into the pathway of blood flow of the parent vessel. The conventional stent might alternatively be placed distally to, but not entirely overlying the circumference of the origin of the side branch to the diseased portion. Such a position of the stent results in a bifurcation treatment that does not provide full coverage or has a gap on the proximal side (the origin of the side branch) of the vessel. It is possible that with a conventional stent having right angled terminal ends could be placed where the entire circumference of the ostium is repaired without compromising blood flow when the bifurcation is formed of a right angle. In such scenarios, extremely precise positioning of the conventional stent is required. This extremely precise positioning of the conventional stent may result with the right angled terminal ends of the conventional stent overlying the entire circumference of the ostium to the diseased portion without extending into a side branch, thereby repairing the right angled bifurcation.

In another method for treating bifurcated vessels, commonly referred to as the “Culotte technique,” the side branch vessel is first stented so that the stent protrudes into the main or parent vessel. A dilatation is then performed in the main or parent vessel to open and stretch the stent struts extending across the lumen from the side branch vessel. Thereafter, a stent is implanted in the side branch so that its proximal end overlaps with the parent vessel. One of the drawbacks of this approach is that the orientation of the stent elements protruding from the side branch vessel into the main vessel is completely random. In addition excessive metal coverage exists from overlapping strut elements in the parent vessel proximal to the carina area. Furthermore, the deployed stent must be re-crossed with a wire blindly and arbitrarily selecting a particular stent cell. When dilating the main vessel the stent struts are randomly stretched, thereby leaving the possibility of restricted access, incomplete lumen dilatation, and major stent distortion.

In yet another procedure, known as “kissing” stents, a stent is implanted in the main vessel with a side branch stent partially extending into the main vessel creating a double-barreled lumen of the two stents in the main vessel distal to the bifurcation. Another approach includes a so-called “trouser legs and seat” approach, which includes implanting three stents, one stent in the side branch vessel, a second stent in a distal portion of the main vessel, and a third stent, or a proximal stent, in the main vessel just proximal to the bifurcation.

The foregoing stent deployment assemblies may encounter some problems and limitations. Typically, there is uncovered intimal surface segments on the main vessel and side branch vessels between the stented segments or there is excessive coverage in the parent vessel proximal to the bifurcation. An uncovered flap or fold in the intima or plaque will invite a “snowplow” effect, representing a substantial risk for sub acute thrombosis, and the increased risk of the development of restenosis. Further, where portions of the stent are left unapposed within the lumen, the risk for subacute thrombosis or the development of restenosis is increased. These stents and delivery assemblies for treating bifurcations are sometimes difficult to use and deliver. Further, even where placement has been successful, the side branch vessel can be “jailed” or covered so that there is impaired access to the stented area for subsequent intervention.

There is sometimes difficulty in treating a diseased bifurcation confined to a vessel segment but extending very close to a distal branch point which is not diseased and does not require treatment. In such circumstances, very precise placement of a stent covering the distal segment, but not extending into the distal side branch, is required.

### SUMMARY OF THE INVENTION

A stent delivery assembly for treating bifurcated vessels having a side branch vessel and a main vessel, comprising a catheter having an expandable member at a distal portion and dual lumens arrange over under extending therethrough, wherein the over lumen includes an open gap or slit extending lengthwise along a side of the catheter and wherein a distal opening of the over lumen is proximal and proximate to the expandable member. A main guide wire passes through the under lumen and into the main vessel. A side branch guide wire passes through the over lumen and out of the distal opening, wherein a wall of the catheter opposite the slit is straight and extends tangentially into the outside diameters of the over and under lumens, wherein the wall of the over lumen has sufficient structural rigidity to restrain the side branch guide wire therein as the catheter is advanced over the main guide wire, and sufficient resilience to enable the side branch guide wire to break out of the over lumen via the slit. A stent for the bifurcation having a side port is disposed over the expandable member, wherein the proximal end of the stent extends at least partially over the distal opening of the over lumen, and the side branch guide wire passes through the stent side port.

In various alternative embodiments, the stent delivery assembly may have an over lumen with a diameter large enough to enable passage of a second stent carried on a second catheter, and/or the over lumen has a diameter large enough to enable passage of a second stent carried on a second catheter. The stent delivery assembly may have an over lumen that has a diameter large enough to enable passage of a second stent carried on a second catheter. Also, the wall of the over lumen may be sufficiently rigid to maintain its shape, avoid structural distortion, and retain the side branch guide wire therein while the catheter is advanced to the bifurcation, and be sufficiently resilient to release the side branch guide wire as it is advanced into the side branch vessel. The over and under lumens may have varying wall thicknesses to control strength of the over lumen with a slit. Also, the catheter wall alongside the over lumen and under lumen may be thicker for increased axial and overall structural strength of the catheter.

These and other advantages of the invention will become more apparent from the following detailed description thereof and the accompanying exemplary drawings.

### BRIEF DESCRIPTION OF THE DRAWINGS

FIGS. 1-5 show the process of deploying a stent at a bifurcation.

FIG. 6 is a side view of an embodiment of the present invention securement feature in a catheter.

FIG. 7 is a cross-sectional view of the catheter taken along line A-A of FIG. 6.

FIG. 8 is a cross-sectional view of an alternative embodiment of the securement feature.

FIGS. 9-12 show the operation of the securement feature from FIG. 6.

### DETAILED DESCRIPTION OF THE PREFERRED EMBODIMENTS

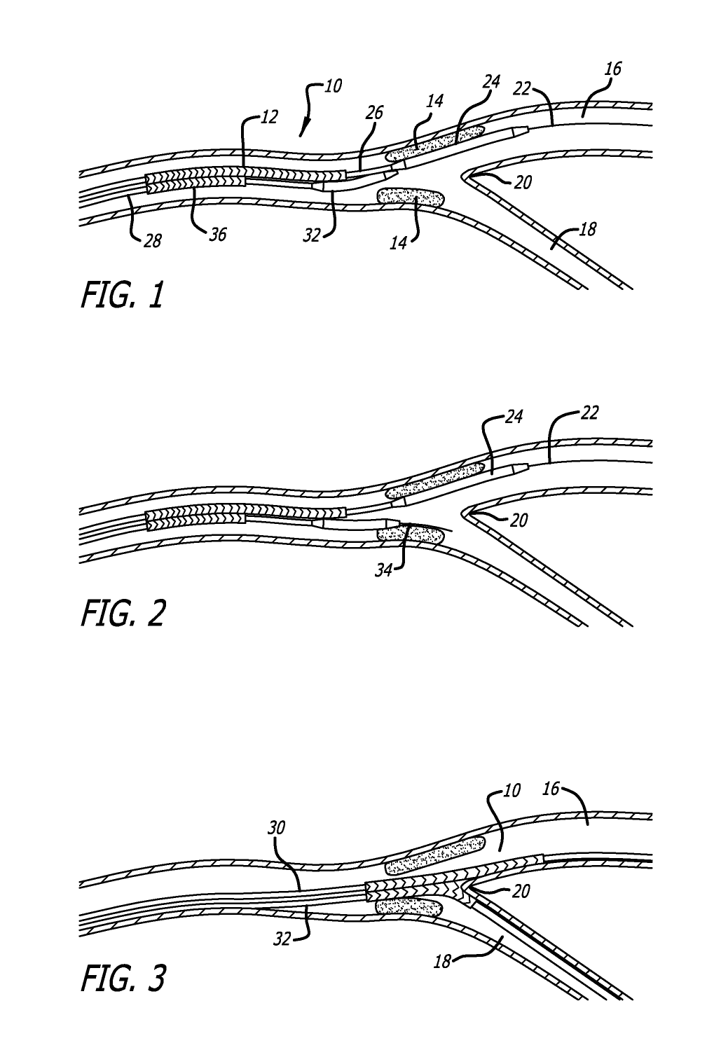

The present invention is directed to a catheter system for treating bifurcation lesions. FIG. 1 is a schematic depiction of a preferred embodiment bifurcated stent delivery system 10 advanced into a patient's bifurcated blood vessel having a main branch vessel 16 and a side branch vessel 18. The bifurcated system 10 is used for treatment of plaque 14 located at the bifurcation. The system 10 includes a dual lumen catheter 30, 32 carrying a specially designed bifurcated stent 12 thereon and having one catheter branch 30 extending the entire length of the stent 12 and another catheter branch 32 passing through a port 36 in the stent 12. The pair of catheter branches 30, 32 at the distal ends are held together by an optional joining mandrel 24 and wherein the main branch guide wire 22 extends into the main branch 16. Each branch of the catheter 30, 32 includes a respective expandable member or balloon 26, 28. The side branch balloon 28 exits the stent 12 at about the midpoint of the stent 12.

The intravascular stent 12 has a pattern or configuration that permits the stent to be tightly compressed or crimped onto a catheter to provide an extremely low profile and to prevent relative movement between the stent and the catheter. The stent also is highly flexible along its longitudinal axis to facilitate delivery through the tortuous intravascular lumens of the patient, but which is stiff and stable enough radially in its expanded condition to maintain the patency of a body lumen such as an artery when the stent is implanted therein.

The stent 12 generally includes a plurality of cylindrical rings that are interconnected to form the stent. The stent is typically mounted on a balloon catheter if it is balloon expandable or mounted on or in a catheter without a balloon if it is self-expanding and made from an alloy such as nitinol (Ni—Ti). In one embodiment, each of the cylindrical rings making up the stent has a proximal end and a distal end and a cylindrical plane defined by a cylindrical outer wall surface that extends circumferentially between the proximal end and the distal end of the cylindrical ring. Generally, the cylindrical rings have a serpentine or undulating shape that includes at least one U-shaped element, and typically each ring has more than one U-shaped element. The cylindrical rings are interconnected by at least one connecting link which attaches one cylindrical ring to an adjacent cylindrical ring. The undulating links are highly flexible and allow the stent to be highly flexible along the stent longitudinal axis. The undulating links may have a curved portion that extends transverse to the stent longitudinal axis. More specifically, the curved portion extends in a transverse direction (or circumferentially) such that it would intersect with the adjacent U-shaped element. The adjacent U-shaped element is preferably shorter in length than other U-shaped elements in the same ring. Thus, when the stent is compressed or crimped onto the catheter, the undulating portion of the links does not overlap or intersect with the adjacent U-shaped element since that element is shorter in length than other U-shaped elements in the same ring. With this structure, the stent can be compressed or crimped to a much tighter or smaller diameter on the catheter, which permits low profile delivery as well as tight crimping force on the catheter to reduce the likelihood of movement between the stent and the catheter during delivery and prior to implanting the stent in the vessel.

A portal region 36 is formed between two adjacent cylindrical rings and is configured to optionally receive a side branch balloon of a balloon catheter. In this embodiment, a first balloon of a balloon catheter extends through the main body of the stent, and a second balloon extends through the portal area so that the two balloons are substantially side by side. The two balloons can be of different lengths and diameters with the first balloon being longer than the second balloon. In this embodiment, three undulating links connect adjacent cylindrical rings. The undulating portion or bend in two of the links of the portal region face in opposite directions transverse to the longitudinal axis of the stent. The undulating links connecting cylindrical rings in the main body of the stent have undulating portions or bends that all point in the same direction transverse to the longitudinal axis of the stent.

The stent 12 may be bare metal or drug coated. Specifically, the stent can be coated with a drug or therapeutic agent to assist in repair of the bifurcated vessel and may be useful, for example, in reducing the likelihood of the development of restenosis. Further, the stent (usually made from a metal) may require a primer material coating to provide a substrate on which a drug or therapeutic agent is coated, since some drugs and therapeutic agents do not readily adhere to a metallic surface. The drug or therapeutic agent can be combined with a coating or other medium used for controlled release rates of the drug or therapeutic agent. Examples of therapeutic agents that are available as stent coatings include rapamycin, ererolimus clobetasol, actinomycin D (ActD), or derivatives and analogs thereof. ActD is manufactured by Sigma Aldrich, 1001 West Saint Paul Avenue, Milwaukee, Wis. 53233, or COSMEGEN, available from Merck. Synonyms of actinopmycin D include dactinomycin, actinomycin IV, actinomycin 11, actinomycin X1, and actinomycin C1. Examples of agents include other antiproliferative substances as well as antineoplastic, antinflammatory, antiplatelet, anticoagulant, antifibrin, antithomobin, antimitotic, antibiotic, and antioxidant substances. Examples of antineoplastics include taxol (paclitaxel and docetaxel). Examples of antiplatelets, anticoagulants, antifibrins, and antithrombins include sodium heparin, low molecular weight heparin, hirudin, argatroban, forskolin, vapiprost, prostacyclin and prostacyclin analogs, dextran, D phe pro arg chloromethylketone (synthetic antithrombin), dipyridamole, glycoprotein, IIb/IIIa platelet membrane receptor antagonist, recombinant hirudin, thrombin inhibitor (available from Biogen), and 7E 3B® (an antiplatelet drug from Centocore). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen), angiotensin converting enzyme inhibitors such as Captopril (available from Squibb), Cilazapril (available from Hoffman LaRoche), or Lisinopril (available from Merck); calcium channel blockers (such as Nifedipine), colchicine fibroblast growth factor (FGF) antagonists, fish oil (omega 3 fatty acid), histamine antagonist, Lovastatin (an inhibitor of HMG-CoA reductase, a cholesterol lowering drug from Merck), monoclonal antibodies (such as PDGF receptors), nitroprusside, phosphodiesterase inhibitors, prostaglandin inhibitor (available from Glazo), Seramin (a PDGF antagonist), serotonin blockers, steroids, thioprotease inhibitors, triazolopyrimidine (a PDGF antagonist), and nitric oxide. Other therapeutic substances or agents which may be appropriate include alpha-interferon, genetically engineered epithelial cells, and dexamethasone.

It should be understood that any reference in the specification or claims to a drug or therapeutic agent being coated on the stent is meant that preferably one or more layers can be coated either directly on the stent or onto a primer material on the stent to which the drug or therapeutic agent readily attaches.

In FIGS. 1-2, the delivery system 10 is advanced to a point just proximal to the target bifurcation. The delivery system 10 includes a stent delivery catheter assembly made from an elongated catheter body which has a proximal catheter shaft, an intermediate section or mid-section, and a distal section. The catheter assembly contains an over-the-wire (OTW) guide wire lumen extending from the proximal catheter hub to one of the distal tips of the distal end of the catheter. The catheter assembly also includes a rapid exchange (Rx) guide wire lumen which extends from the proximal end of the mid-section to one of the distal tips of the distal end of the catheter. The proximal catheter shaft also contains an inflation lumen which extends from the proximal hub of the proximal catheter shaft to the mid-section of the catheter and is in fluid communication with the inflation lumen contained within the mid-section. The mid-section contains lumens for both the OTW and the Rx guide wire lumen. The Rx guide wire lumen begins at about the proximal section of the intermediate shaft and extends to one of the distal tips of the distal catheter shaft. The Rx guide wire lumen may be replaced by a fixed wire design having a fixed guide wire with a distal section permanently secured to a distal section of the catheter branch. The OTW guide wire lumen extends through the intermediate section of the catheter and extends proximally to the catheter hub connected to the proximal catheter shaft and extends distally to one of the tips of the distal section of the catheter.

The distal section of the catheter, shown in FIGS. 1-5, consists of two shafts 30, 32 extending from the distal end of the mid-shaft to the distal end of the catheter tips. Each shaft has a balloon 26, 28 adjacent the distal end followed by a tip connected to the distal end of the balloon. Each shaft contains a guide wire lumen 50, 52 and an inflation lumen (not shown). The inflation lumen of each shaft is in fluid communication with the inflation lumen of the mid-shaft. One of the shafts of the distal section contains an Rx guide wire lumen, which extends proximally through the mid-section of the catheter and exits at about the proximal end of the mid-section of the catheter. The Rx guide wire lumen also extends distally to one of the tips of the distal section of the catheter. The other shaft of the distal section contains an OTW guide wire lumen, which extends proximally through the mid-section and proximal section of the catheter and exits at the proximal hub connected to the distal end of the proximal catheter section. The OTW guide wire lumen also extends distally to one of the tips of the distal section of the catheter.

The distal section of the catheter optionally includes two balloons 26, 28. One balloon is longer and is connected to one of the shafts of the distal catheter section. The long balloon is connected to the catheter shaft such that the inflation lumen of the shaft is in fluid communication with the balloon and the guide wire lumen contained within the shaft extends through the center of the balloon. The proximal section of the balloon is sealed to the distal end of the shaft and the distal end of the balloon is sealed around the outside of the guide wire lumen or inner member running through the center of the balloon. The proximal and distal seals of the balloon allow for fluid pressurization and balloon inflation from the proximal hub of the catheter. The short balloon is connected in the same manner as the long balloon described above to the alternate shaft of the distal section of the catheter. Each balloon has a tip extending from their distal ends. The tips are extensions of the inner members extending through the center of the balloon and contain a lumen for a guide wire associated with each guide wire lumen. The distal end of the catheter has two tips associated with their respective balloons and the guide wire lumen or inner member. One tip is longer and contains the mandrel 24 used for joining the tips during delivery of the previously described stent.

The stent 12 of the present invention is crimped or compressed onto the long balloon 26 and the short balloon 28 such that the long balloon 26 extends through the distal opening and the proximal opening in the stent, while the short balloon 28 extends through the proximal opening and the side port 36 of the stent.

The procedure is, generally, the system is advanced to the bifurcation via the Rx guide wire 22 and into the main branch vessel 16. The joining mandrel 24 is removed by unlocking it at the proximal adapter hub and withdrawing it from the OTW lumen. A new guide wire 34 is then introduced into the OTW lumen to exit the side branch balloon tip and placed in the side branch vessel 18. The system is then advanced into the bifurcation until forward motion stops. With a single inflation device (not shown), both the main and side branch balloons are pressurized, deploying the stent in the main branch and opening a porthole into the side branch.

In the drawings, in FIG. 2, the joining mandrel 24 is retracted, thus releasing the OTW side branch tip. An exchange length guide wire 34 is inserted.

In FIG. 3, the side branch guide wire 34 is positioned into the side branch 18 and the system is advanced to the carina 20.

In FIG. 4, the two balloons 26, 28 are simultaneously inflated using a single indeflator to expand the stent 12 into the main and side branches 16, 18.

In FIG. 5, the balloons 26, 28 are deflated and the catheter system is retracted leaving the deployed stent 12 and the guide wires 22, 34.

As described above, the stent 12 includes a side port 36. The short scaffold of the stent covers the side branch ostium. Radiopaque markers delineate the proximal end, distal end, and location of the side port 36.

As should be understood here, with two guide wires 22, 34 being advanced through the patient's tortuous anatomy, there is a chance of the guide wires twisting. The present invention catheter includes a guide wire securement feature that retains the side branch guide wire proximate to the catheter shaft, thereby reducing the risk of guide wire twisting during advancement. Further, the need for a joining mandrel 24 described above may be eliminated.

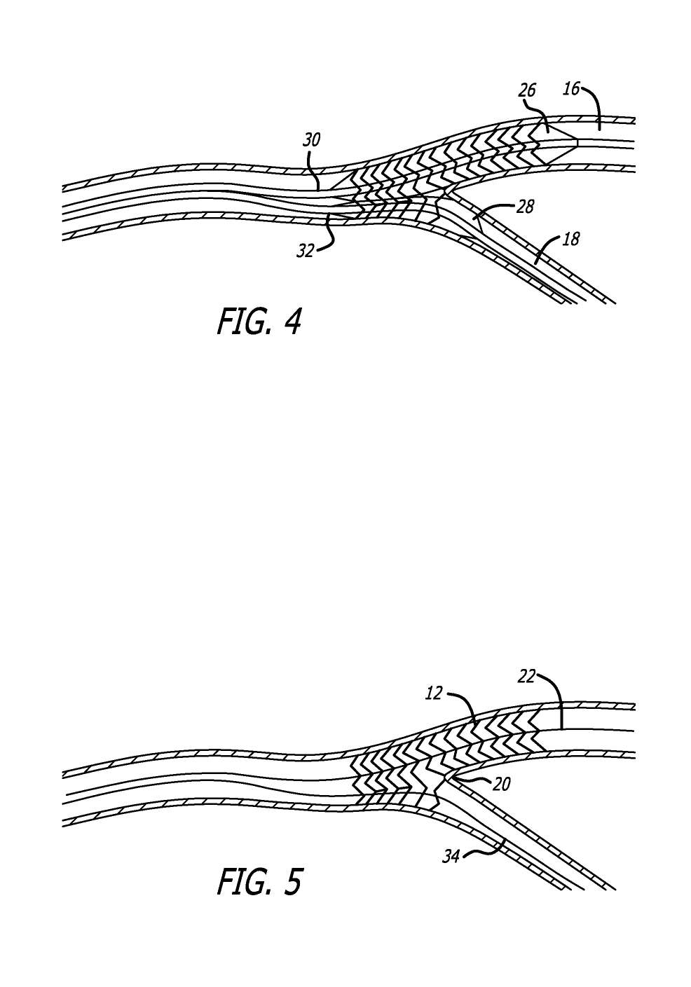

FIG. 6 is a schematic depiction of a preferred embodiment of the present invention catheter design for treating bifurcated vessels. The stent delivery system 10 includes a main branch balloon 42 and stent 44 as described above and also includes a guide wire securement feature, here a hooked or curved flap 38, associated with the catheter shaft proximal to the balloon 42. As shown in FIG. 6, the securement feature 38 may extend underneath the proximal end 58 of the stent 44. In an alternative embodiment (not shown), the securement feature may also not extend underneath the proximal end 58 of the stent 44.

One advantage of the curved flap 38 is to secure the side branch guide wire 34 proximate or immediately next to the catheter shaft. This allows the side branch guide wire tip to be adjacent the exit port 46 of the stent 44 during advancement, and therefore reduces the risk of the guide wires 34, 22, twisting during advancement to the bifurcation. The twisting occurs when a guide wire is run up the guiding catheter into the side branch. Since there is typically a path of at least 100 cm in distance to reach the target lesion, and since the path normally includes a number of bends and angulations, it is possible for the guide wires to become entangled or twisted with each other along their lengths. When this twisting occurs, it can be difficult to then pass a device such as an angioplasty balloon or stent over the twisted guide wires since the twists cause binding or catch points that are barriers to further advancement. This is especially the case with regard to newer devices that are designed specifically to treat bifurcated lesions and that have both main and side branch guide wires passing through separate lumens in the catheter and stent. In such instance, if the guide wires become twisted, it is practically impossible to advance the catheter without using undue force that generally results in the loss of the stent.

An additional advantage of the present invention flap 38 is that it helps prevent flaring of the proximal stent ring during tracking of the system over the guide wires. Since the flap 38 secures the side branch guide wire 34 relatively closely to the catheter body and stent 44, there is thus radial loading on the stent 44, especially at that proximal stent ring, which can contribute to flaring of the stent rings. By controlling the ring flaring, the present invention flap therefore minimizes touching or scraping of the stent against the guiding catheter when it is retracted.

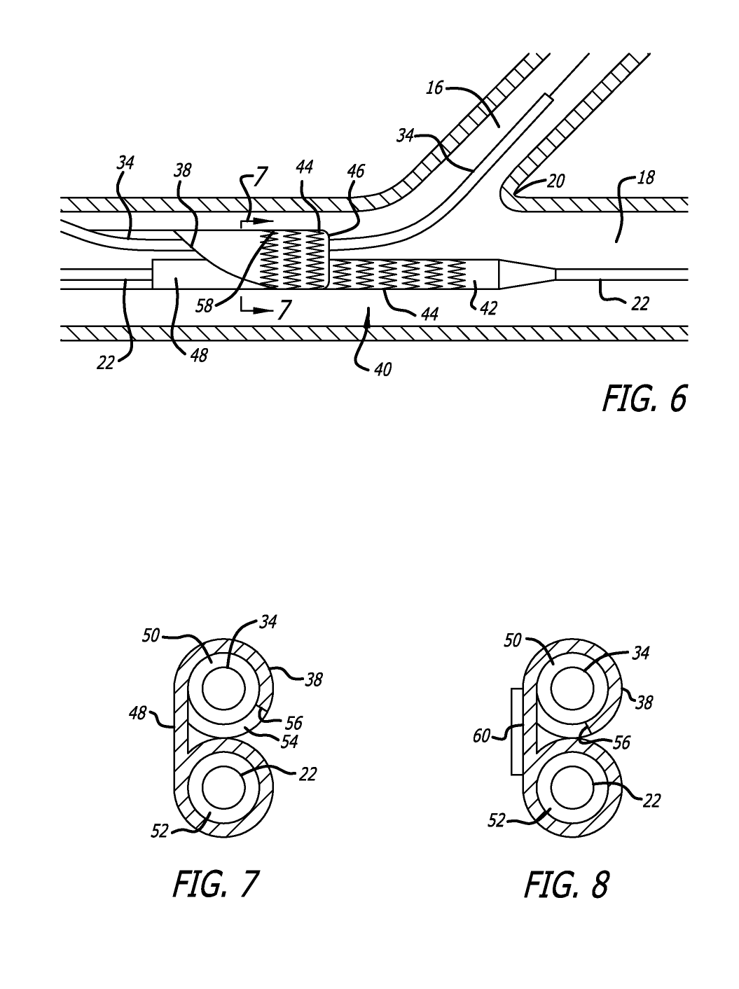

As seen in FIGS. 6-7, the main guide wire 22 and side branch guide wire 34 are arranged in an over-under orientation. The branch guide wire 34 passes through a side port 46 of the stent 44. In FIG. 6, only a main branch balloon 42 is shown. A second balloon catheter may be advanced OTW on guide wire 34, as described above, as needed for treatment. The second balloon catheter and other branch treatment devices can be advanced subsequently over the side branch guide wire 34.

As seen in FIG. 7 which is a cross-sectional view taken along line A-A of FIG. 6, the catheter 48 has an over-under arrangement with the guide wire lumens 50, 52 wherein the respectively guide wires are carried within those lumens. As best seen in FIG. 7, flap 38 is preferably cantilevered from the catheter body and curls or hooks around to create a circular lumen 50 that captures the guide wire 34 therein. The flap 38 preferably circumscribes at least ½ of the outside diameter of the guide wire 34 so that the wire does not unexpectedly escape from the lumen or detach from the catheter. A slit or gap 54 is provided at the leading edge 56 of the flap 38. The preferred embodiment flap 54 has a single leading edge 56 while the rest of the gap region is smooth and curved. This design minimizes the number of cornered edges in the catheter to avoid an exposed edge hanging on any structure.

The flap 54 possesses sufficient rigidity to restrain the side branch guide wire 34 within the lumen 50 yet is sufficiently compliant such that the guide wire may be released and separated from the catheter 48 when located at the carina 20. That is, the flap 38 possesses sufficient structural rigidity to minimize structural distortion and restrain the side branch guide wire 34 within lumen 50 as the catheter is advanced over the main branch guide wire 22 yet has sufficient resilience to enable the side branch guide wire 34 to break out of the lumen 50 via the slit or gap 54.

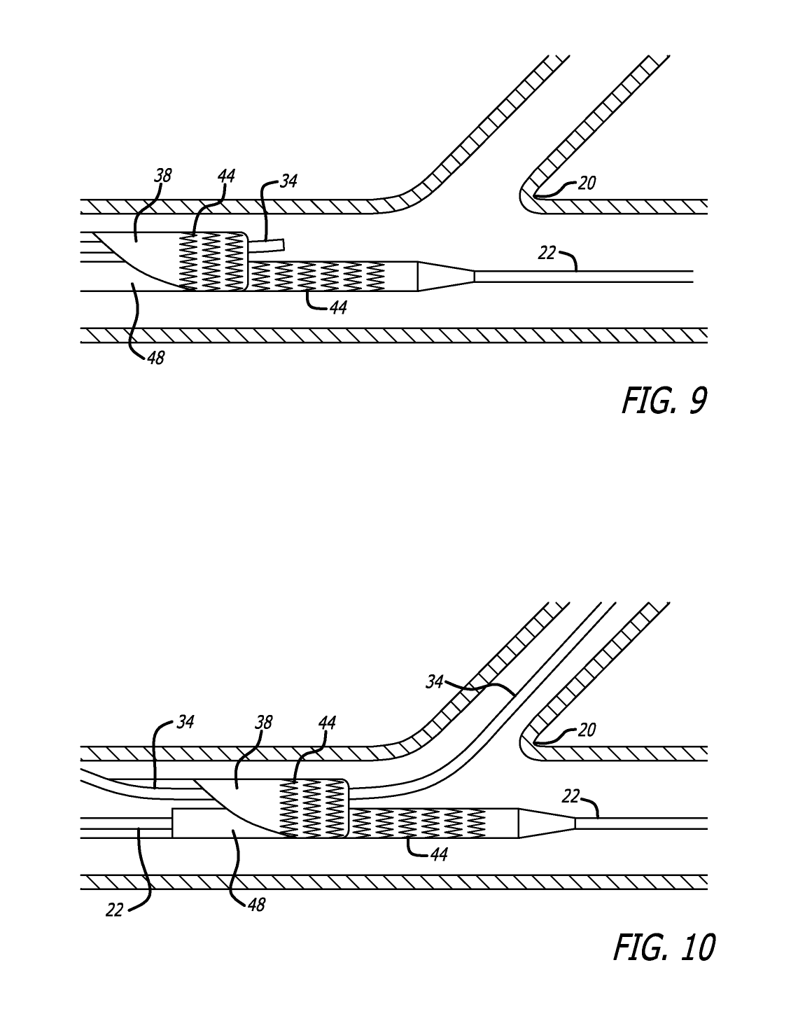

FIG. 8 is a cross-section of an alternative embodiment catheter wherein the flap 38 hooks around the side branch guide wire 34 and the leading edge 56 is proximate to the outside diameter of the under lumen 52. In various alternative embodiments as seen in FIG. 7 versus FIG. 8, the over-under lumens 50, 52 have varying wall thicknesses to control the strength of the over-lumen and its flap 38. The side wall 60 of the catheter 48 may blend tangentially into the outside diameters of the over-under lumens 50, 52 to improve structure rigidity of the catheter due to the missing material at the gap 54 and to enable the proper operation of the flap 38 to release of the secured guide wire 34. With the leading edge 58 curled away in the FIG. 8 embodiment, there is a lower chance of binding or interference with the overlying stent 44 strut pattern during deployment or the patient's blood vessel intima.

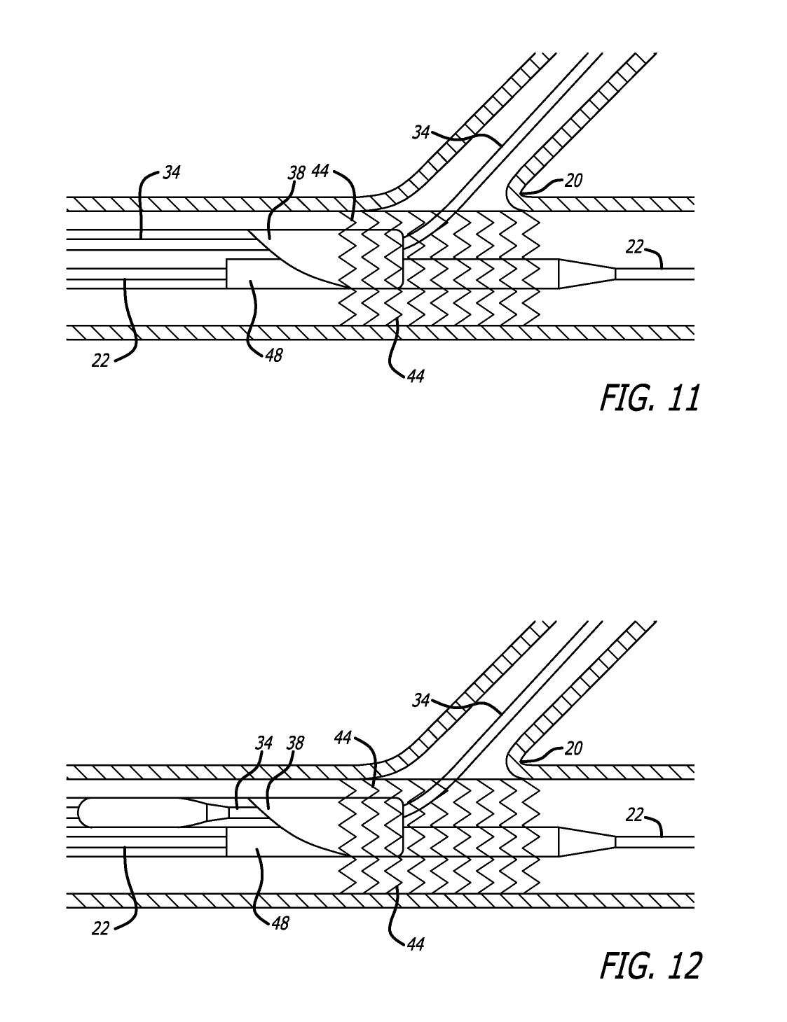

In FIGS. 9-12, the sequence of advancing and deploying the side branch guide wire 34 from the securement feature 38 is depicted. In FIG. 9, the system is advanced along the main guide wire 22 with the side branch guide wire 34 loaded into the flap 38. In FIG. 10, the system is advanced so that the branch guide wire 34 is placed farther into the branch vessel 18. In FIG. 11, the stent 44 has been deployed at the bifurcation.

In FIG. 12, a second balloon catheter has been advanced over the side branch guide wire 34. As the second balloon catheter advances farther into the side branch vessel, the bifurcation tends to split the two guide wires father apart, thus pushing the side branch guide wire 34 out from underneath the flap 38 and freeing the guide wire from the catheter assembly. Once free of the securement flap 38, a branch treatment device such as a second stent (not shown) may be tracked down the wire 34 to enter the side branch vessel 18.

While a particular form of the invention has been illustrated and described, it will also be apparent to those skilled in the art that various modifications can be made without departing from the spirit and scope of the invention. Accordingly, it is not intended that the invention be limited except by the appended claims.

I claim:

1. A stent delivery assembly for treating bifurcated vessels having a side branch vessel and a main vessel, comprising:
a catheter having an expandable member at a distal portion and a main guide wire lumen extending therethrough;
a main guide wire passing through the catheter guide wire lumen and into the main vessel;
a side branch guide wire;
a hooked flap extending from the catheter forming a lumen therein, the flap positioned proximal to the expandable member, wherein the flap forms a longitudinal gap with a leading edge adjacent the catheter, and the side branch guide wire passes through the lumen into the side branch vessel, and wherein the gap size is smaller than the outside diameter of the side branch guide wire, and wherein the flap possesses sufficient rigidity to restrain the side branch guide wire therein as the catheter is advanced over the main guide wire; and
a stent for the bifurcation disposed over the expandable member, wherein the flap at least one of extending underneath the proximal end of the stent and not extending underneath the proximal end of the stent, wherein the stent includes a side port to receive the side branch guide wire therethrough.

2. The stent delivery assembly of claim 1, wherein the flap extends tangentially from an outside diameter of the catheter.

3. The stent delivery assembly of claim 1, wherein the flap extends partially underneath proximal end of stent.

4. The stent delivery assembly of claim 1, wherein the flap distal end is aligned just proximal to stent side port.

5. The stent delivery assembly of claim 1, wherein the hooked flap is a cantilevered extension from the catheter, and captures the side branch guide wire within the lumen.

6. The stent delivery assembly of claim 1, wherein the flap circumscribes over ½ of the guide wire outside diameter.

7. The stent delivery assembly of claim 1, wherein the flap is resilient to flex and release the side branch guide wire as the catheter is advanced into the bifurcation.

8. The stent delivery assembly of claim 1, wherein the gap extends along a side of the catheter.

9. The stent delivery assembly of claim 1, wherein the gap has only one edge.

10. A stent delivery assembly for treating bifurcated vessels having a side branch vessel and a main vessel, comprising:
a catheter having an expandable member at a distal portion and a main guide wire lumen extending therethrough;
a main guide wire passing through the catheter guide wire lumen and into the main vessel;
a side branch guide wire;
a hooked flap extending tangentially from an outside diameter of the catheter forming a lumen and located proximal to the expandable member, wherein the side branch guide wire passes through the lumen into the side branch vessel, and wherein the flap circumscribes an outside diameter of the side branch guide wire and has a single edge proximate to an exterior of the catheter leaving a gap that is smaller than the outside diameter of the side branch guide wire;
wherein the flap possesses sufficient structural rigidity to restrain the side branch guide wire therein as the catheter is advanced over the main guide wire, and sufficient resilience to enable the side branch guide wire to break out of the lumen via the gap; and
a stent for the bifurcation disposed over the expandable member, wherein the flap at least one of extending underneath the proximal end of the stent and not extending underneath the proximal end of the stent, and wherein the stent includes a side port to receive the side branch guide wire therethrough.

11. A stent delivery assembly of claim 10, wherein the distal end of the side branch guide wire is restrained within the hooked flap lumen as the catheter is advanced farther into the bifurcation.

12. A stent delivery assembly of claim 10, wherein the catheter has a circular cross-section and the hooked flap forms a circular cross-section disposed above the catheter with a laterally opening gap.

13. A stent delivery assembly of claim 10, wherein the flap lumen and the main guide wire lumen have an over-under arrangement with the gap opening laterally,

14. A stent delivery assembly for treating bifurcated vessels having a side branch vessel and a main vessel, comprising:
a catheter having an expandable member at a distal portion and dual lumens arrange over under extending therethrough, wherein the over lumen includes an open slit extending lengthwise along a side of the catheter and wherein a distal opening of the over lumen is proximal and proximate to the expandable member;
a main guide wire passing through the under lumen and into the main vessel;
a side branch guide wire passing through the over lumen and out of the distal opening;
wherein a wall of the catheter opposite the slit is straight and extends tangentially into the outside diameters of the over and under lumens;
wherein the wall of the over lumen has sufficient structural rigidity to restrain the side branch guide wire therein as the catheter is advanced over the main guide wire, and sufficient resilience to enable the side branch guide wire to break out of the over lumen via the slit; and
a stent for the bifurcation having a side port, the stent being disposed over the expandable member, wherein the proximal end of the stent extends at least partially over the distal opening of the over lumen, and the side branch guide wire passes through the stent side port.

15. A stent delivery assembly of claim 14, wherein the slit in the over lumen is formed by a curled flap.

16. A stent delivery assembly of claim 14, wherein the stent includes a drug coating.

17. A stent delivery assembly of claim 14, wherein the over lumen includes a diameter large enough to enable passage of a second stent carried on a second catheter.

18. A stent delivery assembly of claim 14, wherein the wall of the over lumen is sufficiently rigid to maintain its shape and retain the side branch guide wire therein while the catheter is advanced to the bifurcation, and sufficient resilient to release the side branch guide wire as it is advanced into the side branch vessel.

19. A stent delivery assembly of claim 14, wherein the over and under lumens have varying wall thicknesses to control strength of the over lumen with a slit.

20. A stent delivery assembly of claim 14, wherein the catheter wall alongside the over lumen and under lumen is thicker for increased axial strength of the catheter.
